(19) United States
(12) Patent Application Publication
(10) Pub. No.: US 2004/0254542 A1
(43) Pub. Date: Dec. 16, 2004

(54) IV apparatus with anti-reflux ball valve

(76) Inventor: John S. Sacco, Fayetteville, NY (US)

Correspondence Address: BERNHARD P. MOLLDREM, JR., 224 HARRISON STREET, SUITE 200, SYRACUSE, NY, 13202, US

(21) Appl. No.: 10/461,521

(22) Filed: Jun. 13, 2003

(51) Int. Cl. A61M 5/00

(52) U.S. Cl. 604/247

(57) ABSTRACT

A gravity flow IV set up for administering fluids intravenously to a patient has a main, low-rate flow branch line and a shunt, high-rate flow branch line, and an auxiliary line above a junction of the main and shunt flow lines which can be used for a second or supplemental source of blood or an IV fluid. In order to ensure that there is no reflux of fluid from one of the main and shunt lines into the other, ball check valves are positioned in each line below the respective drip chamber and above the lower junction of the two lines. The ball check valves can withstand relatively high back pressures without danger of rupture or leakage.

### BACKGROUND OF THE INVENTION

This invention relates to a multi-purpose gravity operated fluid path for administering fluids intravenously to a patient.

Solution administration devices or fluid paths used for delivering intravenous (IV) fluids to a patient are well-known in the art. These devices are sometimes referred to simply as IV sets and generally include a tubular flow line having a capped spike at the upper end that is capable of being inserted into an IV solution bag and a catheter tip at the lower end for infusing fluid into a patient's vein. The flow line also includes a flow regulator, typically in the form of a drip chamber, which regulates flow and establishes the maximum rate of flow that can be passed through the line and thus limits the maximum amount of fluid that will be administered to the patient over a given period of time. One or more adjustable roller clamps are typically attached to the line above and below the drip chamber for either closing off the line completely or partially closing them for further regulating the flow. A graduated burette is generally placed in series flow relationship with the drip chamber to allow the attending health care worker to accurately monitor the amount of fluid administered to the patient.

During most normal procedures, IV fluids are administered continuously over extended periods of time at relatively low flow rates. Oftentimes, however, a situation, such as the need for surgery, will arise where a continuous flow fluid path will not satisfy the needs of the patient. Under these conditions, the normal low-flow administration set-up is removed and replaced with a high-flow set up. When the patient's special needs are satisfied, the high-flow administration set-up is removed and is once again replaced with a new low-flow set-up.

This repeated setting up and taking down of the IV system is a time consuming procedure which wastes substantial amounts of health care time. This loss of time, particularly during emergency procedures, can increase the patient's risk factor. In light of the fact that an IV administration set-up can be used only once, the use of multiple set ups during a single procedure can be costly. More importantly, the hospital must inventory a reasonably large amount of this type of equipment to meet the needs of its patients. Large inventories are space consuming and require a good deal of time and effort to stock and control. The disposal of used administration sets is also causing environmental problems which are now becoming more and more pronounced. Frequent IV starts also increase the risk of infection to the patient and any reduction in the number of starts will be of an immediate benefit to both patient and health care workers alike.

Frequently, a piggy-back arrangement is used to administer medication or blood through the injection port of any intravenous administration set. In this arrangement, a mini-bag, which is attached to a high-flow or low-flow drip chamber, is inserted directly into the main flow line through an injection port located below the primary or low-flow drip chamber. During this procedure, the primary IV bag is lowered and the mini-bag raised to a higher elevation thereby allowing the secondary fluid to be administered by gravity through the injection port. Although this piggy-back arrangement can work well in practice, it nevertheless does have certain disadvantage. The equipment takes time to set up and must be closely monitored, again necessitating excessive use of valuable health care time. Typically, most patients require more than one secondary infusion and, as a result, the main fluid path will be invaded repeatedly. This, of course, increases the risk of infection. The needle used to invade the fluid path also poses a constant danger to the attending health care worker. Unless the health care worker diligently exercises extreme care, the attending worker can puncture him- or herself with the needle during the injection procedure. The initial wound itself may not be dangerous; however, puncture wounds provide a means by which blood-borne infections can be acquired. Such wounds require the hospital worker himself or herself to seek medical attention, which also consumes valuable health care resources.

In my earlier U.S. Pat. No. 5,059,173, I disclosed a branched IV administration set-up that includes a capped spike for receiving an IV bag at the top end thereof and a needle unit at the bottom end thereof for injecting fluids into a patient. A main flow line has a first drip chamber mounted therein which is capable of administering fluids at a first flow rate. A shunt line is placed in parallel with the main flow line to bypass the first drip chamber. A second drip chamber is mounted in the shunt line that is capable of administering fluids at a second flow rate that is significantly higher than the rate through the main line. Clips and/or clamps are used to selectively open and close the lines to route IV fluids through a selected one of the two available drip chambers. An auxiliary line may also be provided above the junction of the main flow line and the shunt line. The auxiliary line also contains a capped spike at its proximal end for receiving a second IV fluid bag. Additional clips and/or clamps are contained in the lines for selectively connecting one of the two available IV bags in fluid flow communication with the main flow line. There are one-way valves disposed in the main and shunt lines ahead of the lower connector and below the respective drip chambers. The purpose of the one-way valve is to prevent fluid reflux, i.e., to prevent the fluid that is flowing in one of the two flow lines from flowing upward into the other flow line. This prevents mixing of the fluids in the drip chamber.

In the existing IV apparatus, the one-way mechanism in the valve is simply a rubber diaphragm. The diaphragm will block reverse flow so long as the pressures involved are relatively low. However, in the event that a blood bag is installed onto the auxiliary line for injection of blood or plasma into the patient, the pressures involved can typically reach 150 to 300 Torr or possibly higher. In such cases, there is a risk that the rubber diaphragm in the one-way valve could rupture, and still permit reverse flow of the blood or other fluid back up into the low-flow-rate drip chamber. In such case, the blood, flowing through the shunt line, could contaminate the fluid flowing in the main flow line. This can pose a risk to the patient, and can be a patient safety issue. In addition, if fluid reflux of this type occurs, then the IV apparatus has to be removed and replaced, which once again consumes valuable health care resources. Accordingly, a need exists to improve patient safety and medical care effectiveness by reducing the risk of fluid reflux.

### OBJECTS AND SUMMARY OF THE INVENTION

It is therefore an object of the present invention to improve flow paths used to administer IV fluids to a patient.

A further object of this invention is to conserve valuable health care time when administering IV fluids.

A still further object of the present invention is to lessen the patient's risk when undergoing medical procedures involving the administration of IV fluids.

Another object of the present invention is to reduce the amount of equipment required to administer IV fluids to a patient who may require frequent changes in medication and dosages.

It is yet another object of the present invention to reduce the cost involved in administering IV fluids to a patient.

Still another object of the present invention is to reduce the amount of IV equipment that must be disposed of by a health care facility.

A further object of the present invention is to reduce the amount of inventory that must be kept on hand by a health care facility.

Yet a further object of the present invention is to protect health care workers from potentially dangerous needle punctures by reducing the number of times an existing flow path must be invaded by a needle.

A still further object of the present invention is to provide an attending physician with greater flexibility when administering IV fluids to a patient.

Another object of the present invention is to reduce the number of IV starts to safely satisfy a patient's needs.

An important object of this invention is to reduce the risk of fluid reflux in the IV set-up.

These and other objects of the present invention are attained by means IV apparatus defining a gravity flow fluid path for administering fluids intravenously to a patient. The apparatus has an upper portion with penetration means at its top end for coupling to a source of fluid such as an IV fluid bag, and a lower portion with injection means at its bottom end for introducing the fluid into the patient. A Y-connector or similar upper junction joins the upper portion to a main flow line that includes a mini-drip chamber for administering fluids at a medically low rate, and to a shunt flow line that includes an adult drip chamber for administering fluids at a medically high flow rate. A lower junction, e.g., Y-connector joins lower ends of the main flow line and the shunt flow line to the lower portion of the gravity flow fluid path. Clips or similar control means on the main flow line and shunt flow line are operative to selectively route one or two fluids respectively through the main and shunt flow lines. An auxiliary line is connected to the upper portion of ahead of the upper junction, and includes means for introducing fluid from a fluid container into the gravity flow fluid path. A pair of one-way check valves are situated respectively in the main flow line below the mini-drip chamber and in the shunt flow line below the adult drip chamber to prevent fluid flowing through one of the main and shunt flow lines from moving upwardly in the other of the flow lines. In the embodiments of this invention, at least one of these one-way check valves comprises a ball check valve including a housing, a ball within the housing and formed of a medical grade plastic material, means within the housing for establishing a limited vertical freedom of motion for the ball such that the ball normally permits downward flow of fluid therethrough, but in the event of a reverse fluid pressure the ball seals against an upper portion of the housing. Optionally, a spring applies an adjustable spring force onto the ball.

The ball check valve easily withstands reverse pressures of several hundred Torr without any danger of rupture or leakage, so that there is no reflux of blood or other fluids from the shunt line into the drip chamber of the main line.

For a better understanding of these and other objects of the present invention, reference will be made to the following detailed description of the invention which is to be read in conjunction with the accompanying Drawing.

### BRIEF DESCRIPTION OF THE DRAWINGS

FIG. 1 is a side elevation showing a gravity operated flow path for administering IV fluids to a patient which embodies the teachings of the present invention, and

FIG. 2 is a sectional view taken along lines 2-2 in FIG. 1.

FIG. 3 is a sectional elevation of the improved one-way valve according to the present invention.

### DETAILED DESCRIPTION OF THE PREFERRED EMBODIMENT

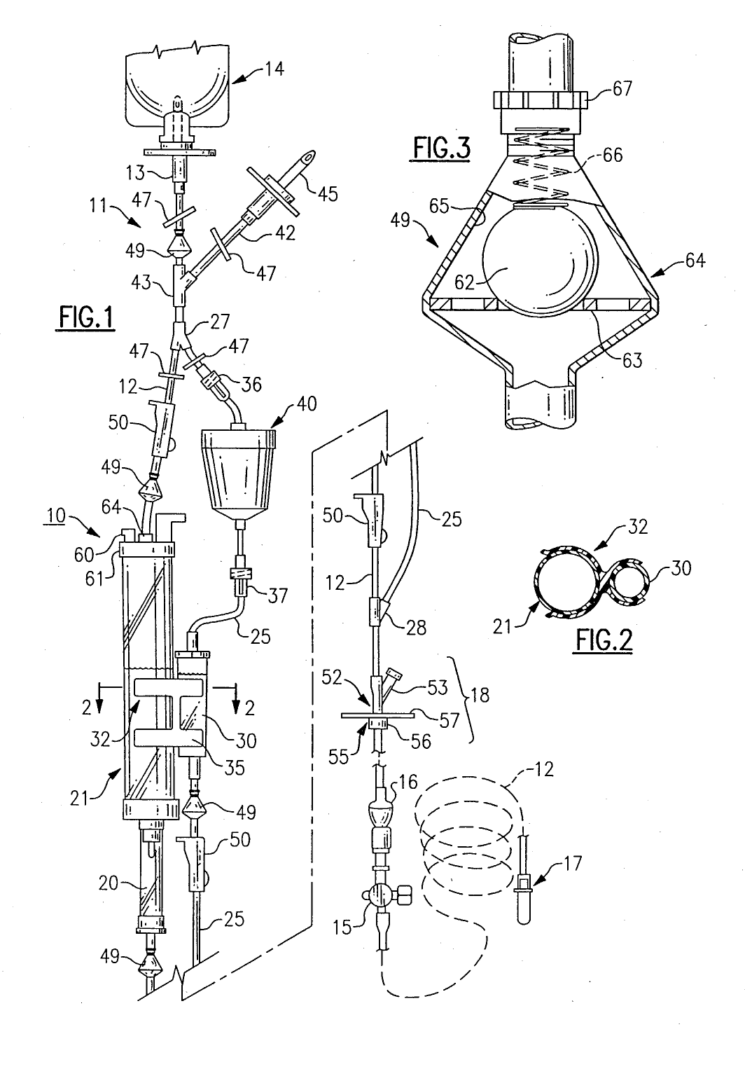

Referring now to the Drawing, and initially to FIG. 1 thereof, a gravity-operated flow path, generally referenced 10, is presented which embodies the teachings of the present invention. The flow path is made up of sections of flexible, clear plastic tubing. A top end portion 11 of the flow path is equipped with a capped spike 13 of conventional design that is capable of penetrating an IV fluid bag 14 thereby enabling the fluid contained in the bag to freely enter the main flow line. Although not shown, the IV fluid bag is furnished with a strap by which the bag, and thus the flow path set-up, is suspended from a suitable hanger or IV pole. The bag is supported at an elevated position so that the IV fluids contained therein can flow under the influence of gravity downwardly through the flow path 10. A lower end portion 18 of the flow path 10 is equipped with a three-way stopcock 15, a bubble flush 16, and a catheter tip 17, all of which are conventional devices that are well-know and used in the art. An upper Y-connector 27 bifurcates the flow path 10 into a main flow line 12 and a shunt flow line 25. A lower Y-connector 28 joins the lower ends of the main flow line 12 and shunt flow line 25 to the lower end portion 18.

A first primary drip chamber 20 is mounted in the main flow line 12 that serves to regulate the rate of fluid flow that can pass through the line and thus be administered to the patient. In this embodiment of the invention, The chamber 20 is a mini-drip chamber, which administers IV fluids to the patient at a relatively low flow rate continuously over a long period of time. The mini-drip chamber is placed in series with a burette 21 that has a vertically disposed graduated scale (not shown) which allows an attending health care worker to accurately monitor the patient's fluid intake. Drip chamber and burette combinations of this type are sold commercially by Kendall McGraw, Inc. of Sabana Grande, Puerto Rico under the trade name Metriset.

The shunt flow line 25 is placed in parallel with the main flow line 12 to provide an alternate flow path around the mini-drip chamber and burette combination. The shunt line 25 is made up of lengths of flexible, clear plastic tubing. The shunt line is connected to the upper end portion 11 by the Y-connector 27 which is situated above the burette and to the lower end portion 18 by the connector 28 situated below the mini-drip chamber. A secondary “adult” drip chamber 30 is mounted on the shunt line and is arranged to pass fluids at a relatively higher flow rate than the mini-drip chamber. In this embodiment of the invention, the adult drip chamber 30 is supported on the larger burette 21 by means of a resilient plastic support member 32, shown in section in FIG. 2. The support member 32 can be conveniently snapped over both the adult drip chamber and the burette to hold the two in parallel vertical alignment as shown in FIG. 1. The support member can also be slidably positioned along the length of the burette to selectively change the elevation of the adult drip chamber, as required.

A Luer lock connector having a male member 36 and a female member 37 is placed in the shunt line above the adult drip chamber. The Luer lock connector permits the shunt line to be separated so that a blood filter 40 or other IV related device can be operatively connected into the shunt line. As will become apparent from the disclosure below, the shunt line can be employed in the present system to rapidly infuse blood or selected medications into a patient without having to replace or disconnect the IV set-up. The Luer lock arrangement also allows the blood filter to be periodically changed without disturbing the existing administration set-up. Typically, blood filters require changing after the administration of one to two units of blood. When fluids, other than blood, are being administered through the shunt line, the blood filter can be removed and the line rejoined by simply coupling together the two members 36, 37 of the Luer lock connector.

An auxiliary line 42 is also connected into the flow path by means of Y-connector 43 located above the connector 27 that separates the main line 12 and the shunt line 25. The upper tip of the auxiliary line is also equipped with a second capped spike 45. By use of the capped spike 45, a unit of blood or a second IV bag (not shown) can be coupled in fluid flow communication with the main flow line or shunt flow line. Shut-off clips 47-47 are mounted in the main line 12 and the shunt flow line 25 just below the connector 27, and in the auxiliary line 42 and the upper portion 11 directly above the connector 43. The clips can be operated to open one line and close the other so that fluids from a selected one of the two available IV bags can be routed into the shunt flow line or the main flow line, as needed. A one-way valve 49 is placed in the main line above connector 43 to prevent fluids from auxiliary line 42 from backing up into the upper section of the main line.

The shut-off clips 47-47 mounted in the main line and the shunt line immediately below connector 27 can be selectively opened and closed to route IV fluids from one of the two available IV bags into either the main line or the shunt line. Accordingly, the attending health care worker, at his or her option, can select one of the two available fluids for administration, and additionally the desired administration rate can be selected without having to beak down or invade existing set-ups. Accordingly, changes and dosages in fluids can be made rapidly and safely. This, of course, reduces the risk to the patient and reduces the amount of IV equipment needed to satisfy a patient's needs during various procedures. In short, the present apparatus provides an immediate benefit to everyone in the health care chain, including patients, medical workers, and care facility administrators.

One-way valves 49 are positioned in the flow lines immediately below each drip chamber 21, 30 to prevent fluids from moving upwardly in the lines and thus possibly mixing one fluid with another. In most cases, this reverse flow is not a problem because of the gravity flow arrangement. However, the valve will provide for added safety during the administration of fluids. Adjustable roller clamps 50-50 are also mounted at strategic positions within the main flow line and the shunt flow line. These roller clamps are operable to further control the flow of fluids through the lines or alternatively shut down the lines completely as may be required.

An injection port 52 is mounted in the main flow line directly below the lower Y-connector 28. The injection port contains a rubber-tipped arm 53 through which a needle can be inserted thereby allowing further fluids to be introduced directly into the main flow line for rapid infusion into the patient. As noted above, it is not uncommon for a health care worker to puncture him or herself with the needle while attempting to pass in into the injection port. This is particularly true in emergency situations where time is important. The present device is provided with a molded circular shield 55 that surrounds the main flow line directly below the injection port. The shield includes a hub 56 that embraces the flow line without crimping it and a radially expanded flange 57 mounted upon the hub. When injecting a fluid into the injection port, the health care worker simply grasps the line below the shield with one hand, and passes the needle through the rubber tip port with the other hand. In the event the needle slips from the port, it will strike the expanded flange 57, rather than the worker's hand, thus protecting the worker from a potentially dangerous puncture wound.

The apparatus of the present invention can also be adapted to simultaneously administer two separate fluids through the catheter. In this operation, the burette 21 is filled with a desired amount of a first fluid and the shunt line 25 is arranged to administer a second fluid from one of the available IV bags. The clamp 50 in the main flow line below the burette/drip chamber combination is then opened to allow the first fluid to be administered along with the second fluid. The ball check valves 49 prevent upward fluid flow and keep the two IV fluids from entering the opposite flow path.

The burette contains an air vent 60 mounted in the top wall 61 thereof which can be selectively opened when the main drip chamber is in use. Although not shown, a flapper valve is also located over the lower outlet of the burette which automatically closes when the liquid in the burette becomes depleted thereby preventing air from entering the mini-drip chamber. With the air vent opened a positive head pressure is exerted upon the fluid in the burette to provide for an ever and continuous flow of fluid through the mini-drip chamber. As noted above, the two strategically placed one-way check valves 49-49 positioned beneath each drip chamber permits non-competitive gravity-fed flow to be maintained for two different types of fluids. Accordingly, various combinations of fluids such as medications, blood, IV solution and the like can be brought to the IV catheter at junction 28 at various desired flow rates.

The adult drip chamber 30 can be used for the rapid infusion of fluids when required by the health care worker. There are set up arrangements, therefore, where air can be drawn through the air vent 60 upwardly through line 12, connector 27 and thus pass into the shunt line 25, thus posing a problem. To avoid this problem, one of the one-way check valves 49 is placed in the primary or main flow line 12 immediately above the inlet 64 to the burette. The valve functions to permit fluid to flow under the influence of gravity from the supply bag or bottle, into the burette, but prevents fluids, including air, from moving in the opposite direction. Accordingly, air is thus prevented from being drawn into the shunt line.

As mentioned above, when a blood bag or plasma bag is placed on the auxiliary line 42, the fluid may be subjected to higher pressures than those experienced in normal operations. More specifically, it is common to squeeze the blood bag so as to increase the pressure for rapid influx of blood into the patient. This can result in pressures of 300 Torr, or possibly higher, which can be present at the connector 28 that joins the lower ends of the adult or shunt flow line 25 to the main line or low-flow line 12. In order to ensure that the one-way check valve 49 can withstand the relatively high pressures without danger of rupture, the usual diaphragm valve of the prior art is replaced here with a ball check valve 49, as shown in FIG. 3.

The check valve 49 has a ball 62 contained within housing 64 that includes a ball chamber 65 that is generally conic in shape. The ball 62 is favorably a resilient medical grade plastic. A lower seat 63 defines a lower limit to movement of the ball, and permits downward flow of fluid around the ball 62 and seat 63. The conic wall 65 of the ball housing 64 defines an upper limit to movement of the ball, and engages the ball 62 to seal it when the ball moves up with reverse or upward fluid flow. An optional coil spring 66 is shown here biasing the ball 62 open, and provides a limited resistance to upward movement of the ball. An upper end of the spring 66 is coupled to a finger wheel 67, and the latter can be rotated to adjust the spring force. The ball 62 will easily withstand the pressures of several hundred Torr that can be experienced when a quantity of blood or other fluid has be injected into the patient by way of the auxiliary line 42. Of course, while the ball member shown in this embodiment is spherical in shape, equivalent valves can employ ball members of other suitable shapes, which will serve to wedge into the valve housing and resist or block reverse (upward) fluid flow.

While this invention has been explained with reference to the structure disclosed herein, it is not confined to the details as set forth and this application is intended to cover any modifications and changes as may come within the scope of the following claims.

I claim:

1. In a gravity flow fluid path for administering fluids intravenously to a patient, in which an upper portion has penetration means at its top end for coupling to a source of fluid, a lower portion has injection means at its bottom end for introducing said fluid into the patient, an upper junction joins said upper portion to a main flow line that includes a mini-drip chamber for administering fluids in said main flow line at a medically low rate, and to a shunt flow line that includes an adult drip chamber for administering fluids passing in said shunt flow line at a medically high flow rate, a lower junction joins lower ends of said main flow line and said shunt flow line to the lower portion of said gravity flow fluid path, control means on said main flow line and said shunt flow line are operative to selectively route one or two fluids respectively through the main and shunt flow lines, an auxiliary line connected to said upper portion ahead of said upper junction includes means for introducing fluid from a fluid container into said gravity flow fluid path, and a pair of one-way check valves are situated respectively in the main flow line below the mini-drip chamber and in the shunt flow line below the adult drip chamber to prevent fluid flowing through one of the main and shunt flow lines from moving upwardly in the other of the flow lines; the improvement in which at least one of said one-way check valves comprises a ball check valve including a housing, a ball within said housing, means within said housing for establishing a limited vertical freedom of motion of said ball such that the ball normally permits downward flow of fluid therethrough, but in the event of a reverse fluid pressure said ball seals against an upper portion of said housing.

2. The gravity flow fluid path according to claim 1, wherein said ball housing has a generally conic ball chamber.

3. The gravity flow fluid path according to claim 1, wherein the ball check valve further includes a spring biasing said ball downwards.

4. The gravity flow fluid path according to claim 3, wherein the ball check valve further includes means for adjusting a spring force of said spring.

5. The gravity flow fluid path according to claim 1, wherein said ball is formed of a medical grade resilient plastic material.
